(19) United States
(12) Patent Application Publication
(10) Pub. No.: US 2005/0182342 A1
(43) Pub. Date: Aug. 18, 2005

(54) Monitoring fluid flow in the gastrointestinal tract

(75) Inventors: David A. Dinsmoor, St. Paul, MN (US); Mark J. Traffas, Lakeville, MN (US)

Correspondence Address: MEDTRONIC, INC., 710 MEDTRONIC PARKWAY NE, MS-LC340, MINNEAPOLIS, MN, 55432-5604, US

(73) Assignee: Medtronic, Inc., Minneapolis, MN (US)

(21) Appl. No.: 10/835,425

(22) Filed: Apr. 29, 2004

Related U.S. Application Data
(60) Provisional application No. 60/544,611, filed on Feb. 13, 2004.

(51) Int. Cl. A61B 5/103; A61B 5/117

(52) U.S. Cl. 600/593; 600/549; 73/170.01; 600/300

(57) ABSTRACT

A sensor capable of sensing the flow of fluids is placed in the gastrointestinal tract. The sensor may be, for example, an ultrasonic flow sensor, an optical flow sensor, or a thermal convection flow sensor. A system for monitoring fluid flow in the gastrointestinal tract may include monitor configured for placement in the gastrointestinal tract that includes such a sensor. The monitor measures the flow of fluid in the gastrointestinal tract based on the output of the senor. The monitor may take the form of a capsule with a means or mechanism for attachment to a mucosal lining of the gastrointestinal tract. In exemplary embodiments, the sensor is placed in the esophagus, senses the flow of fluid from the stomach into the esophagus, and is used to diagnose gastroesophageal reflux disease (GERD).

This application claims the benefit of U.S. Provisional Application Ser. No. 60/544,611, filed Feb. 13, 2004, the entire content of which is incorporated herein by reference.

### FIELD OF THE INVENTION

The invention relates to medical devices and methods and, more particularly, to medical devices and methods for measuring fluid flow within the gastrointestinal tract.

### BACKGROUND

Gastroesophageal reflux occurs when stomach fluid, which typically includes stomach acids, intermittently flows from the stomach into the esophagus. It is common for most people to experience this fluid reflux occasionally as heartburn. Gastroesophageal reflux disease (GERD) is a clinical condition in which the reflux of stomach fluid into the esophagus is frequent enough and severe enough to impact a patient's normal functioning or to cause damage to the esophagus.

In the lower part of the esophagus, where the esophagus meets the stomach, there is a muscular valve called the lower esophageal sphincter (LES). Normally, the LES relaxes to allow food to enter into the stomach from the esophagus. The LES then contracts to prevent stomach fluids from entering the esophagus. In GERD, the LES relaxes too frequently or at inappropriate times, allowing stomach fluids to reflux into the esophagus.

The most common symptom of GERD is heartburn. Acid reflux may also lead to esophageal inflammation, which causes symptoms such as painful swallowing and difficulty swallowing. Pulmonary symptoms such as coughing, wheezing, asthma, or inflammation of the vocal cords or throat may occur in some patients. More serious complications from GERD include esophageal ulcers and narrowing of the esophagus. The most serious complication from chronic GERD is a condition called Barrett's esophagus in which the epithelium of the esophagus is replaced with abnormal tissue. Barrett's esophagus is a risk factor for the development of cancer of the esophagus.

Accurate diagnosis of GERD is difficult but important. Accurate diagnosis allows identification of individuals at high risk for developing the complications associated with GERD. It is also important to be able to differentiate between gastroesophageal reflux, other gastrointestinal conditions, and various cardiac conditions. For example, the similarity between the symptoms of a heart attack and heartburn often lead to confusion about the cause of the symptoms.

Esophageal manometry, esophageal endoscopy, and esophageal pH monitoring are standard methods of measuring esophageal exposure to stomach acids and are currently used to diagnose GERD. Table 1 below lists documents that disclose techniques for diagnosing or detecting GERD, and other documents that disclose techniques for measuring luminal flow.

TABLE 1

| Patent | | |
|---|---|---|
| Number | Inventors | Title |
| 5,479,935 | Essen-Moller | Ambulatory Reflux Monitoring System |
| 5,833,625 | Essen-Moller | Ambulatory Reflux Monitoring System |
| 5,967,986 | Cimochowski et al. | Endoluminal Implant with Fluid Flow |
| | | Sensing Capability |
| 5,967,989 | Cimochowski et al. | Ultrasonic Sensors for Monitoring the |
| | | Condition of a Vascular Graft |
| 6,285,897 | Kilcoyne et al. | Remote Physiological Monitoring |
| | | System |
| 6,398,734 | Cimochowski et al. | Ultrasonic Sensors for Monitoring the |
| | | Condition of Flow Through a |
| | | Cardiac Valve |
| 6,585,763 | Keilman et al. | Implantable Therapeutic Device |
| | | and Method |
| 6,689,056 | Kilcoyne et al. | Implantable Monitoring Probe |

All documents listed in Table 1 above are hereby incorporated by reference herein in their respective entireties. As those of ordinary skill in the art will appreciate readily upon reading the Summary of the Invention, Detailed Description of the Preferred Embodiments and Claims set forth below, many of the devices and methods disclosed in the patents of Table 1 may be modified advantageously by using the techniques of the present invention.

### SUMMARY OF THE INVENTION

In general, the invention is directed to techniques for monitoring fluid flow in the gastrointestinal tract. In some embodiments, a system according to the invention monitors the reflux flow of fluid from the stomach into the esophagus. In such embodiments, the system may be used to diagnose gastroesophageal reflux disease (GERD).

Various embodiments of the present invention provide solutions to one or more problems existing in the prior art with respect to prior techniques for detecting and diagnosing GERD. These problems include the inability of prior techniques to reliably diagnose GERD in particular situations. In particular, rather than directly measure the reflux flow of fluid from the stomach into the esophagus, the prior techniques measure the secondary effects of the reflux flow. However, these secondary effects are not apparent, detectable, or present in all GERD cases.

For example, one prior technique for diagnosing GERD involves visually inspecting the mucosal lining of the esophagus via esophageal endoscopy. However, in some patients experiencing reflux flow of fluid from the stomach to the esophagus, the mucosal lining of the esophagus is not yet damaged or visibly damaged such that GERD would be diagnosed via esophageal endoscopy. A term used to describe these situations is endoscopy negative reflux disease. If GERD is not diagnosed in such situations and the reflux flow persists, the patients may experience significant discomfort and additional damage to the mucosal lining of the esophagus prior to being diagnosed with GERD.

Other example prior techniques for diagnosing GERD involve measuring the acidity level, i.e., the pH, of fluid in the esophagus. However, in some patients the fluid flowing from the stomach into the esophagus is not sufficiently acidic such that GERD can be diagnosed by pH measurement. In such patients, other components of the stomach fluid, such as bile and digestive enzymes, may cause the mucosal damage and the other symptoms associated with GERD. Again, if GERD is not diagnosed in such situations, the patients may experience significant discomfort and additional damage to the mucosal lining caused by the reflux flow of stomach fluid.

Various embodiments of the present invention are capable of solving at least one of the foregoing problems. When embodied in a system for monitoring the flow of fluid in the gastrointestinal tract, for example, the invention includes various features such as a sensor capable of sensing the flow of fluid. The sensor may output a signal as a function of at least one of velocity and rate of fluid flow, and may be, for example, an ultrasonic flow sensor, an optical flow sensor, or a thermal convection flow sensor.

In some embodiments, a system according to the invention includes a monitor configured for placement in the gastrointestinal tract, e.g., the esophagus, that includes such a sensor. In such embodiments, the monitor measures the flow of fluids in the gastrointestinal tract based on one or more signals output by the sensor. The monitor may store the flow measurements for later retrieval. In other embodiments, the system may include a receiver external to the patient and the monitor may transmit flow measurement information to the external receiver for storage and/or processing. The monitor may transmit flow measurement information to the receiver wirelessly via inductive coupling between the monitor and the external receiver. The information stored within the monitor and/or the receiver may be downloaded by a clinician to a computing device and analyzed to diagnose the condition of the patient.

The monitor may take the form of a capsule that includes a housing, and the sensor may be located within, may be integral with, may protrude from, or may be mounted on the housing. In such embodiments, the sensor may sense fluid flow outside of the housing. The capsule may include any of a variety of means and/or structures for attaching the capsule to a mucosal lining of the gastrointestinal tract, such as the mucosal lining of the esophagus. In some embodiments, the system includes a delivery device, which may be an endoscopic delivery device, comprising a handle and a flexible probe that extends from the handle into the gastrointestinal tract of the patient. In such embodiments, the capsule is coupled to a distal end of the probe for delivery to an attachment site within the gastrointestinal tract.

In comparison to known techniques for diagnosing maladies of the gastrointestinal tract, various embodiments of the invention may provide one or more advantages. For example, various embodiments of the invention provide a sensor within the esophagus capable of detecting the flow of fluid from the stomach into the esophagus. As such, the invention may provide more reliable diagnosis of GERD through the monitoring of the reflux flow itself rather than the secondary effects thereof. Additionally, the invention may provide earlier diagnosis of GERD through the monitoring of reflux flow, e.g., before symptoms of GERD are visible via endoscope. Further, according to some embodiments of the invention, the signal is processed to determine the direction of fluid flow. By determining the direction of fluid flow, the system is advantageously able to distinguish between fluid flow associated with swallowing in a direction toward the stomach, and reflux fluid flow from the stomach into the esophagus.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 is a schematic diagram illustrating a gastrointestinal fluid flow monitoring system shown in conjunction with a patient.

FIG. 2 is a cross-sectional schematic diagram illustrating a monitor of the gastrointestinal fluid flow monitoring system of FIG. 1.

FIG. 3 is a block diagram illustrating the monitor of FIG. 2.

FIG. 4 is a schematic diagram further illustrating the gastrointestinal fluid flow monitoring system of FIG. 1 as including a delivery device for positioning and placing a monitor within the gastrointestinal tract.

FIGS. 5(A)-(D) are cross-sectional schematic diagrams illustrating placement of a monitor.

FIG. 6 is a flow diagram illustrating an example technique for monitoring fluid flow within the gastrointestinal tract.

### DESCRIPTION OF THE PREFERRED EMBODIMENTS

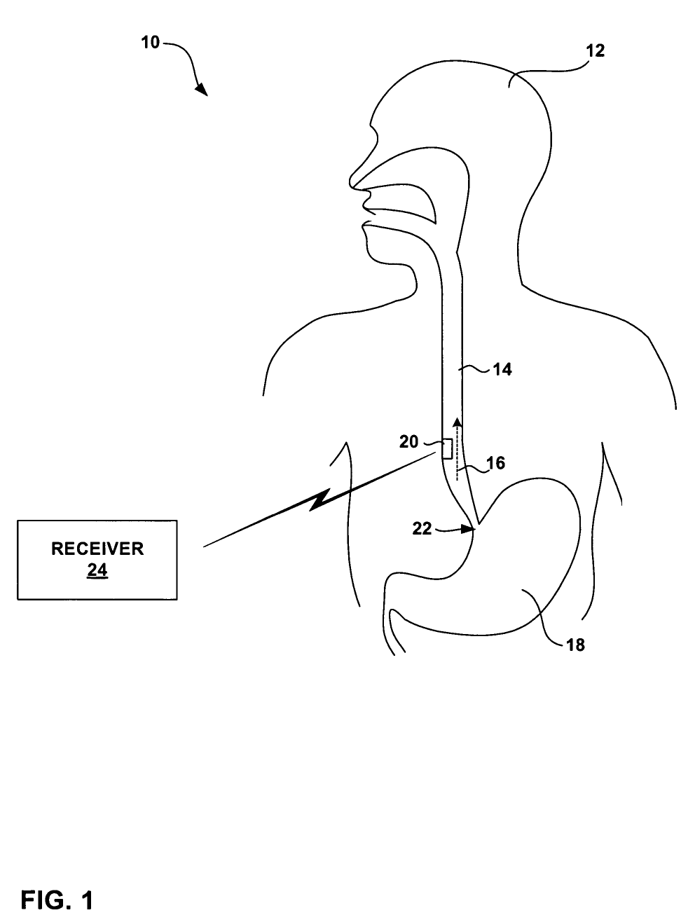

FIG. 1 is a schematic diagram illustrating a gastrointestinal fluid flow monitoring system 10 shown in conjunction with a patient 12. In the illustrated embodiment, fluid flow monitoring system 10 monitors the flow of fluid within the lower portion of an esophagus 14 of patient 12. More specifically, fluid flow monitoring system 10 monitors the flow of fluid in the reflux direction, indicated by arrow 16, from a stomach 18 of patient 12 into the lower portion of esophagus 14. Monitoring the reflux flow of fluid from stomach 18 into the lower portion of esophagus 14 allows a clinician to more accurately diagnose Gastroesophageal Reflux Disease (GERD).

System 10 includes a monitor 20 positioned within esophagus 14 near the lower esophageal sphincter (LES) 22 of patient 12, i.e., where esophagus 14 meets stomach 18. As described above, LES 22 normally relaxes to allow food to enter into stomach 18 from esophagus 14. LES 22 then contracts to prevent stomach acids from entering esophagus 14. In patient 12 experiencing GERD, LES 22 relaxes too frequently or at inappropriate times, allowing fluid to reflux from stomach 18 into the esophagus 14, which may lead to complications such as heartburn, painful swallowing, difficulty swallowing, coughing, wheezing, asthma, inflammation of the vocal cords or throat, esophageal ulcers, narrowing of the esophagus, and in the worst cases Barrett's esophagus.

Monitor 20 includes a sensor that is capable of sensing the flow of fluid, and monitors the reflux flow of fluid 16 from stomach 18 into esophagus 14 based on a signal generated by the sensor as a function of the flow of fluid. Monitor 20 may detect occurrences of reflux flow, may periodically measure the reflux flow, or a combination thereof. As will be described in greater detail below, monitor 20 may take the form of a capsule that is attached to the mucosal lining of esophagus 14, and may monitor reflux fluid flow outside of a housing of the capsule.

In the illustrated embodiments, system 10 also includes a receiver 24 in wireless communication with monitor 20. In particular, monitor 20 transmits flow information, such as indications of flow events or flow measurements, to receiver 24 via any of a variety of telemetry techniques known in the art. Monitor 20 may include a transmitter (not shown), and both monitor 20 and receiver 24 may include an antenna (not shown) to facilitate transmittal of flow information from monitor 20 to receiver 24. Receiver 24 may, for example, comprise a portable receiver that is carried by patient 12, e.g., a pager-like device that may be attached to a belt or carried within a pocket of patient 12 and includes a patch antenna that may be attached to the skin of patient 12 over monitor 20.

Receiver 24 may store the information received from monitor 20, and in some embodiments may process the information. Receiver 24 may include a user interface, e.g., a keypad and display, and may display flow information received from monitor 20 to patient 12. In such embodiments, frequent transmission of flow information from monitor 20 to receiver 24, e.g., every 12 seconds, may be preferred. Receiver 24 may also allow patient 12 to mark the time of the occurrence of events, e.g., symptoms such as heartburn or vomiting, via the keypad.

The information stored within receiver 24 may be downloaded by a clinician to a computing device and analyzed to diagnose the condition of patient 12. The computing device may process the information to provide the clinician with a variety of useful representations thereof. For example, timing diagrams indicating flow events, patient-marked events, and/or measured flow over time may be presented. As other examples, mean or median measured flow values, or histograms with number of flow events, or mean or median measured flow values for various time bins may be presented. A flow event may be a measured flow greater than a threshold value.

System 10 may be used to monitor reflux flow 16 for a period of time, e.g., 24-48 hours, as part of a study to enable a clinician to diagnose GERD. Monitor 20 may eventually self-detach from the lining of esophagus 14, e.g., due to the lining sloughing off or use of a biodegradable mechanism for attaching monitor 20 to the lining, and is passed through the gastrointestinal tract of patient 12. Further details regarding the use of an esophageal monitor and receiver to collect information for presentation to a clinician and diagnosis of GERD may be found in the incorporated Kilcoyne et al. patents (U.S. Pat. No. 6,285,897 and U.S. Pat. No. 6,698,056).

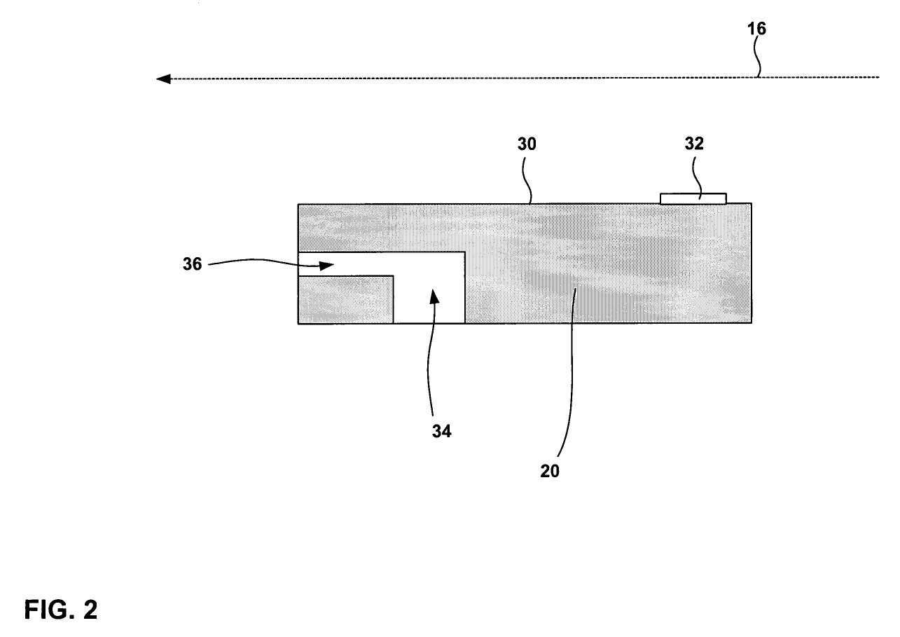

FIG. 2 is a cross-sectional schematic diagram illustrating monitor 20. In the illustrated embodiment, monitor 20 takes the form of a capsule and includes a generally capsule-shaped housing 30. Housing 30 may comprise one or more biocompatible materials, such as silicones, plastics, polytetrafluoroethylene (PTFE), ceramics, stainless steel, or titanium. Monitor 20 also includes a sensor 32 capable of sensing the flow of fluid outside of housing, i.e., within the gastrointestinal tract of patient 12. Sensor 32 may output a signal as a function of one or both of the velocity or rate of fluid flow. Sensor 32 may be located within, may be integral with, may protrude from, or may be mounted on housing 30.

In some embodiments, sensor 32 comprises an ultrasonic flow sensor, and may include one or more transducers, such as piezoelectric crystals, to convert electrical energy to acoustical energy. Sensor 32 may include one or more transducers to emit acoustical energy, and one or more transducers to receive acoustical energy. In some embodiments, sensor 32 may comprise a pulsed Doppler ultrasonic sensor in which a single transducer emits acoustical energy as pulses and receives acoustical energy of the pulses reflected by flowing fluid. Doppler shifting of the frequency of the reflected energy indicates the velocity of the fluid flow. Consequently, in some embodiments, monitor 20 may include circuitry, such as a quadrature phase detector, in order to enable monitor 20 to distinguish the direction of the flow of fluid in addition to its velocity.

In some embodiments, sensor 32 comprises a laser Doppler flow sensor, and may include a laser emitter and a photodiode to detect laser light as reflected by the fluid flow. Again, monitor may include circuitry, such as a quadrature phase detector, in order to enable monitor 20 to distinguish the direction of the flow of fluid in addition to its velocity.

In other embodiments, sensor 32 may include any one or more of a thermal-convection velocity sensor, e.g., including a thermistor, an AC or DC electromagnetic flow sensor, a sensor that senses the concentration of a natural or introduced component of the stomach fluid, or a temperature sensor. A thermal-convection velocity sensor 32 may include a heating element upstream of the thermistor to heat fluid within the esophagus such that flow rate may be measured according to the temperature of the heated fluid when it arrives at the thermistor. In other embodiments, flow rate may be determined from the output of a concentration or temperature sensor using Fick's techniques.

However, in dye or thermodilution flow sensing embodiments, a dye or cold saline may be required to be delivered to stomach 18. In such embodiments, system 10 may include an indwelling catheter or other delivery mechanism for periodically or continuously delivering the dye or cold saline. Further, in embodiments in which the concentration of a natural stomach fluid component is measured in esophagus, the amount of the component within the stomach 18 may be measured during placement of monitor 20 or estimated based on an average patient.

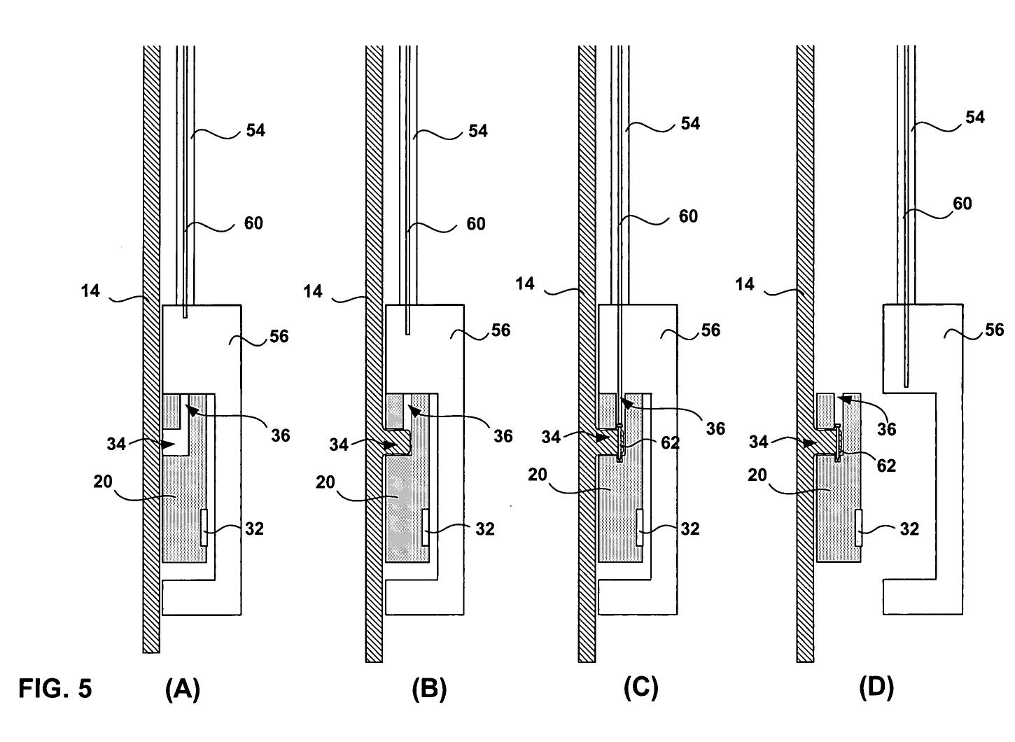

In some embodiments, as illustrated in FIG. 2, housing 30 defines a chamber 34 and a vacuum outlet 36. In such embodiments, chamber 34 and outlet 36 facilitate attachment of housing 30 to the mucosal lining of the gastrointestinal tract, as will be described in greater detail below with reference to FIGS. 5(A)-(D).

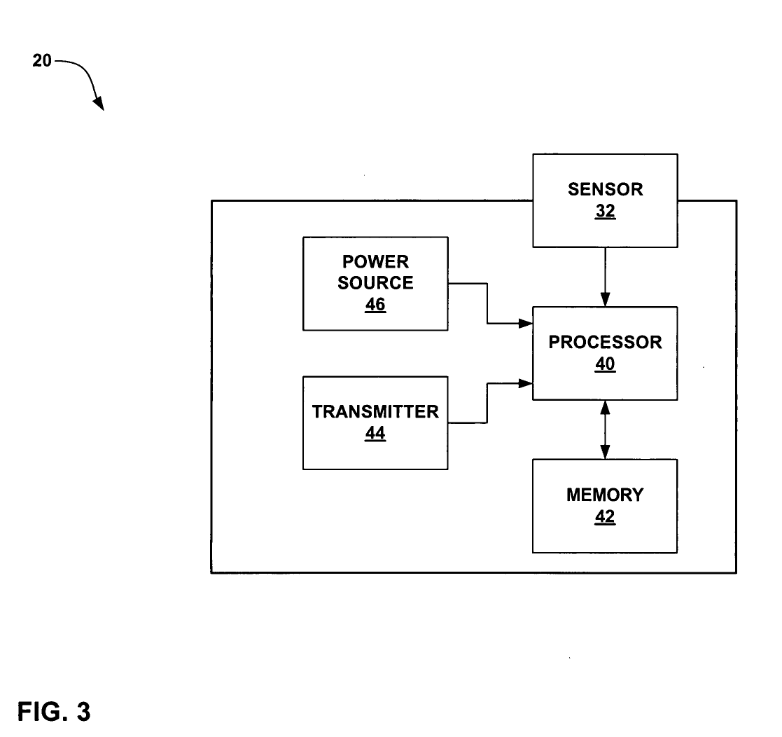

FIG. 3 is a block diagram illustrating monitor 20. Monitor 20 includes a processor 40 that receives one or more signals from sensor 32, and monitors fluid flow within the gastrointestinal tract based on the signals. Processor 40 may store indications of flow events and/or flow velocity and/or rate measurements within a memory 42, and may use one or more threshold values stored in memory 42 to identify flow events.

Processor 40 may also process the signal received from sensor 32 to determine a direction of fluid flow, or may also receive a signal indicating the direction of the fluid flow. For example, the signal output by sensor 32 may be processed by a quadrature phase detector, which may output a signal to processor 40 indicating the direction of fluid flow. Processor 40 may monitor reflux flow of fluid from stomach 18 to esophagus 14 based on the direction of the fluid flow. Specifically, by determining the direction of fluid flow, the processor 40 is advantageously able to distinguish between fluid flow associated with swallowing in a direction toward the stomach, and reflux fluid flow from the stomach into the esophagus, i.e., in a retrograde direction within the esophagus.

Although depicted in FIG. 3 as including a single sensor 32, monitor 20 may include a plurality of sensors 32, which may be located at a variety of positions on or within housing 30. Processor 40 may process signals from multiple sensors 32 to more accurately monitor fluid flow. For example, processor 40 may average or otherwise combine the signals to ameliorate inaccuracies in the measurement of fluid flow attributed to the position of any one sensor 32 on or within housing. In some embodiments where direction of fluid flow is monitored, sensor 32 may be of a type wherein a single sensor does not provide direction information, such as a thermal-convection velocity sensor, an electromagnetic flow sensor, a concentration sensor, or a temperature sensor. In such embodiments, processor 40 may compare the signals of multiple sensors 32 of one of these types to determine the direction of fluid flow, e.g., to monitor reflux flow of fluid from stomach 18 into esophagus.

Processor 40 may include one or more microprocessors, digital signal processors (DSPs), application-specific integrated circuits (ASICs), field-programmable gate arrays (FPGAs), and/or other digital logic circuitry. Memory 42 may include any magnetic, electronic, or optical media, such as random access memory (RAM), read-only memory (ROM), electronically-erasable programmable ROM (EEPROM), flash memory, or the like. Memory 42 may store program instructions that, when executed by processor 40, cause processor 40 to perform the functions ascribed to it herein.

As shown in FIG. 3, monitor 20 may also include a transmitter 44 and a power source 46. Transmitter 44 may be coupled to an antenna (not shown) and, as described above, processor 40 may transmit flow information determined based on the signal from sensor 32 and stored in memory 44 to receiver 24 (FIG. 1) via transmitter 44 and the antenna.

Power source 46 provides power for the other components 32 and 40-44 of monitor 20, and may include a battery or capacitor, e.g., a super capacitor. In some embodiments, power source 46 is rechargeable via induction or ultrasonic energy transmission, and includes an appropriate circuit for recovering transcutaneously received energy. For example, power source 46 may include a secondary coil and a rectifier circuit for inductive energy transfer. In other embodiments, power source 46 may not include any storage element, and monitor 20 may be fully powered via transcutaneous inductive energy transfer. The energy may be provided to monitor 20 by receiver 24.

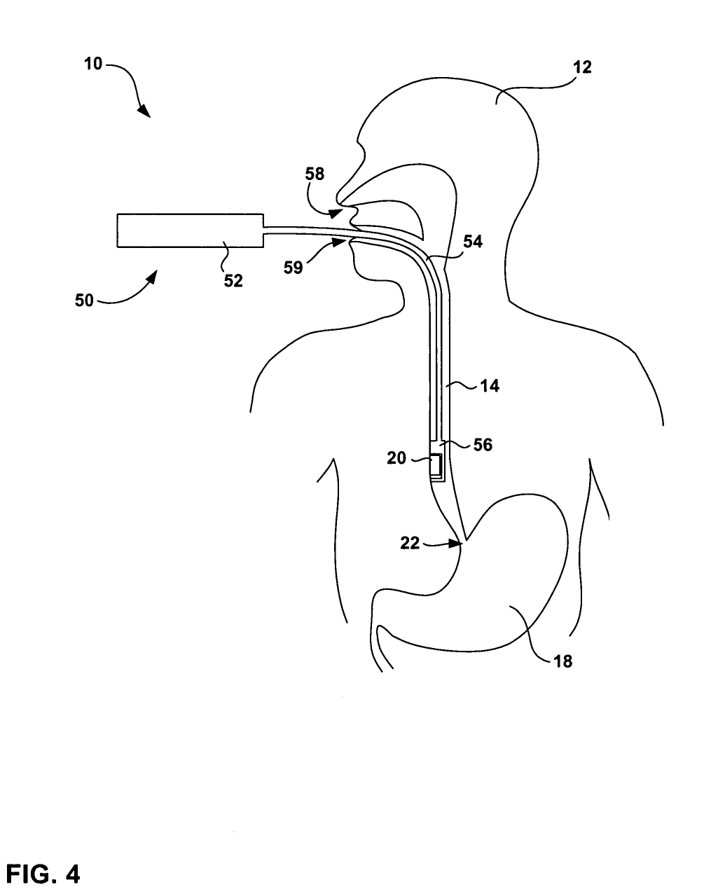

FIG. 4 is a schematic diagram further illustrating system 10 as including a delivery device 50 for guiding monitor 20 to an attachment site within the gastrointestinal tract of patient 12, and attaching monitor 20 to the mucosal lining of the gastrointestinal tract at the attachment site. Delivery device 50 may be an endoscopic delivery device. Delivery device 50 includes a proximal portion, referred to herein as a handle 52, and a flexible probe 54 that extends from handle 52 into the gastrointestinal tract of patient 12. Monitor 20 is coupled to a distal end 56 of delivery device 50 for delivery to an attachment site within the gastrointestinal tract. In the illustrated embodiment, monitor 20 is depicted as being placed at a location within esophagus 14 of patient 12 proximate to LES 22.

In particular, distal end 56 of delivery device 50 enters esophagus 14, via either nasal cavity 58 or oral cavity 59, and extends through esophagus 14 to a desired attachment site. Monitor 20 is attached to the mucosal lining of esophagus 14 at the attachment site, as will be described in greater detail below, and the distal end 56 of delivery device 50 releases monitor 20. For example, capsule 18 can be attached to the lining of esophagus 14 approximately 2 centimeters (cm) above LES 22.

FIGS. 5(A)-(D) are cross-sectional schematic diagrams illustrating placement of monitor 20 according to an embodiment of the invention. Delivery device 50 (FIG. 4) includes a vacuum inlet (not shown) on handle 52 to couple delivery device 50 to a vacuum (not shown). The vacuum applies suction within an inner lumen formed by probe 54. As illustrated in FIG. 5(B), a vacuum outlet 36 at the interface between probe 54 and housing 30 of monitor 20 applies the suction from the vacuum to the lining of esophagus 14 in order to draw esophageal tissue into void 34 within housing 30 of monitor 20.

Delivery device 50 attaches monitor 20 to the esophageal tissue drawn into void 34. As shown in FIG. 5(C), delivery device 22 may, for example, include an advancing shaft 60 to advance an attachment mechanism 62 through the esophageal tissue drawn into void 34 to attach monitor 20 to the lining of esophagus 14. Advancing shaft 60 may be coupled to a plunger (not shown) provided on handle 52 of delivery device 50 (FIG. 4) that allow a clinician to advance the attachment mechanism 62 and attach monitor 20 at the desired location. FIG. 5(D) illustrates the detachment of monitor 20 from delivery device 50, and the removal of delivery device 50 from esophagus 14.

In some embodiments, monitor 20 may be released from attachment to the lining of esophagus 14 to be excreted by patient 12 when the lining of sloughs off. In other embodiments, attachment mechanism 62 may be biodegradable, and monitor 20 may be released when attachment mechanism 62 degrades. In the embodiment illustrated in FIGS. 5(A)-(D), attachment mechanism 62 takes the form of a locking pin, which may be biodegradable. However, any mechanism or means for attachment of monitor 20 to the lining of a gastrointestinal tract may employed in various embodiments of the invention, e.g., included on or attached to housing 30 of monitor 20, including barbs, sutures, or glue. Such attachment mechanisms may be biodegradable. Further, some attachment mechanisms according to the invention do not require application of a vacuum to gastrointestinal tissue or the inclusion of chambers 34 and 36 within the housing 30. Additional details regarding the illustrated techniques and alternative techniques for attaching monitor 20 to a lining of a gastrointestinal tract may be found in the incorporated Kilcoyne et al. patents (U.S. Pat. No. 6,285,897 and U.S. Pat. No. 6,698,056).

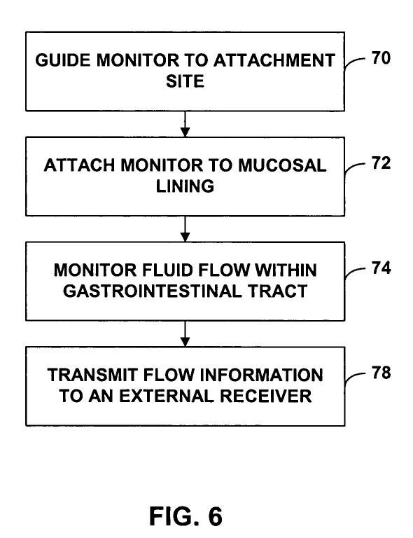

FIG. 6 is a flow diagram illustrating an example technique for monitoring fluid flow within the gastrointestinal tract. A monitor 20 including at least one sensor 32 that senses fluid flow is guided to an attachment site within the gastrointestinal tract of a patient 12 (70). For example, a delivery device 50 may carry monitor 20 through the esophagus 14 of the patient 12 to an attachment site within the esophagus 14 proximate to the LES 22 of the patient, as described above. The monitor 20 is then attached to the mucosal lining of the gastrointestinal tract at the attachment site by any of the techniques described above (72).

Once attached to the gastrointestinal tract, the monitor 20 monitors the flow of fluid within the gastrointestinal tract, e.g., outside of a housing 30 of monitor 20 (74). Monitoring fluid flow may include detection of the occurrence of fluid flow, e.g., detection of flow events, and/or measurement of the fluid flow. As described above, a processor 40 of the monitor 20 may processes one or more signals received from a sensor or sensors 32 of the monitor 20 to monitor the fluid flow. Further, the processor 40 may determine flow direction from the one or more signals, e.g., identify fluid flow in one or more directions, such as identifying fluid flow in a retrograde direction within the esophagus. In some embodiments, the processor 40 monitors reflux flow of fluid from the stomach 18 into the esophagus 14 based on the direction of the fluid flow indicated by the sensor signals. In such embodiments, monitor 20 and/or receiver 24 is advantageously able to distinguish between fluid flow associated with swallowing in the direction toward the stomach 18, from reflux fluid flow in the retrograde direction 16 (FIG. 1) from stomach 18 into esophagus 14. Fluid flow information collected by monitor 20 may be transmitted to an external receiver 24 for use by a clinician in diagnosing maladies of the gastrointestinal tract, such as GERD, as described above (78).

The preceding specific embodiments are illustrative of the practice of the invention. It is to be understood, therefore, that other expedients known to those skilled in the art or disclosed herein may be employed without departing from the invention or the scope of the claims. For example, the invention is not limited to monitoring of reflux fluid flow or esophageal fluid flow, or to diagnosis of GERD. In various embodiments, a sensor 32 for measuring fluid flow may be located anywhere within the gastrointestinal tract, and may measure the flow of fluid in any one or more directions.

For example, a sensor 32, e.g., carried by a monitor 20, may be positioned within the colon to detect fluid flow associated with impending incontinence or diarrhea. In such embodiments, the system 10 may provide an alarm to alert the patient of the impending incontinence or diarrhea, which may be located within the monitor 20 or a receiver 24. In various embodiments, a monitor according to the invention may be used for monitoring, providing alerts or alarms, or feedback control for a delivered therapy

A medical monitor 20 according to the invention is not limited to the described and illustrated capsule-like form. Instead, a monitor 20 may take any of a variety of forms suitable for positioning within the gastrointestinal tract. In some embodiments, for example, a monitor 20 may take the form of a stent or cuff that includes a sensor 32. In other embodiments, a stent or cuff may carry a capsule-like monitor, and may serve to at least temporarily maintain the monitor at a location within a gastrointestinal tract.

In some embodiments, a system 10 does not include a receiver 24. Instead, stored flow information may retrieved directly from monitor 20 when excreted by patient 12, or wirelessly transmitted directly to a clinician computer, e.g., via the Internet or the public switched telephone network (PTSN). Further, in some embodiments, a system 10 includes a plurality of monitors 20, which may be located at various positions within the gastrointestinal tract, or no monitor 20. In some embodiments, a sensor 32 is positioned within the gastrointestinal tract via a catheter, such as an indwelling nasopharyngeal catheter, which carries one or more electrical conductors to couple the sensor 32 to a monitor or computer. Further, the invention is not limited to embodiments in which the sensor 32 is located within the gastrointestinal tract, but instead includes embodiments in which the sensor senses fluid flow within the gastrointestinal tract through the wall thereof.

In the claims, means-plus-function clauses are intended to cover the structures described herein as performing the recited function and not only structural equivalents but also equivalent structures. Thus, although a nail and a screw may not be structural equivalents in that a nail employs a cylindrical surface to secure wooden parts together, whereas a screw employs a helical surface, in the environment of fastening wooden parts a nail and a screw are equivalent structures.

Many embodiments of the invention have been described. Various modifications may be made without departing from the scope of the claims. These and other embodiments are within the scope of the following claims.

1. A method for monitoring reflux flow of fluid from a stomach of a patient into an esophagus of the patient comprising:
receiving a signal from a flow sensor, wherein the sensor is included as part of a medical monitor located within the esophagus that includes a housing, and the sensor outputs the signal as a function of at least one of velocity and rate of fluid flow within the esophagus outside of the housing;
determining a direction of the fluid flow within the esophagus based on the signal; and
monitoring the reflux flow of fluid from the stomach into the esophagus based on the signal and the direction of the fluid flow.

2. The method of claim 1, wherein the sensor comprises an ultrasonic flow sensor.

3. The method of claim 1, wherein the sensor comprises an optical flow sensor.

4. The method of claim 1, wherein the sensor comprises at least one of an electromagnetic flow sensor, a temperature sensor, and a sensor that senses concentration of a component of the fluid.

5. The method of claim 1, wherein determining a direction of the fluid flow comprises identifying fluid flow in a first direction, and monitoring reflux flow of fluid comprises monitoring reflux flow of fluid based on the identification of fluid flow in the first direction.

6. The method of claim 5, further comprising determining at least one of a velocity and a rate of the fluid flow in the first direction, wherein monitoring reflux flow of fluid comprises monitoring reflux flow of fluid based on the at least one of the velocity and the rate of the fluid flow in the first direction.

7. The method of claim 5, wherein fluid flow in the first direction comprises fluid flow in a retrograde direction within the esophagus.

8. The method of claim 7, further comprising identifying fluid flow within the esophagus in a second direction that is toward the stomach, wherein monitoring reflux flow of fluid comprises monitoring reflux flow of fluid based on the identification of fluid flow in the second direction

9. The method of claim 8, further comprising determining at least one of a velocity and a rate of the fluid flow in the second direction, wherein monitoring reflux flow of fluid comprises monitoring reflux flow of fluid based on the at least one of the velocity and the rate of the fluid flow in the second direction.

10. The method of claim 1, wherein the monitor comprises a capsule.

11. The method of claim 1, further comprising:
guiding the monitor to an attachment site within the esophagus; and
attaching the monitor to a mucosal lining of the esophagus at the attachment site.

12. The method of claim 1, wherein the monitor is located proximate to a lower esophageal sphincter of the patient.

13. The method of claim 1, wherein monitoring reflux flow of fluid comprises measuring reflux flow of fluid based on the signal.

14. The method of claim 1, further comprising transmitting flow information to a receiver located outside a body of the patient.

15. A medical monitor comprising:
a housing;
a flow sensor to output a signal as a function of at least one of velocity and rate of fluid flow outside of the housing; and
a processor to determine a direction of the fluid flow based on the signal, and monitor reflux flow of fluid from a stomach to an esophagus based on the direction of the fluid flow and the signal.

16. The monitor of claim 15, wherein the sensor comprises an ultrasonic flow sensor.

17. The monitor of claim 15, wherein the sensor comprises an optical flow sensor.

18. The monitor of claim 15, wherein the sensor comprises at least one of an electromagnetic flow sensor, a temperature sensor, and a sensor that senses concentration of a component of the fluid.

19. The monitor of claim 15, further comprising a quadrature phase detector that receives the signal from the flow sensor and outputs another signal based on the signal received from the flow sensor, wherein the processor determines the direction of the fluid flow based on the signal output by the quadrature phase detector.

20. The monitor of claim 15, wherein the processor identifies fluid flow in a first direction based on the signal, and monitors reflux flow of fluid based on the identification of fluid flow in the first direction.

21. The monitor of claim 20, wherein the processor determines at least one of a velocity and a rate of the fluid flow in the first direction based on the signal, and monitors reflux flow of fluid based on the at least one of the velocity and the rate of the fluid flow in the first direction.

22. The monitor of claim 20, wherein fluid flow in the first direction comprises fluid flow in a retrograde direction within the esophagus.

23. The monitor of claim 22, wherein the processor identifies fluid flow within the esophagus in a second direction that is toward the stomach based on the signal, and monitors reflux flow of fluid based on the identification of fluid flow in the second direction.

24. The monitor of claim 23, wherein the processor determines at least one of a velocity and a rate of the fluid flow in the second direction based on the signal, and monitors reflux flow of fluid based on the at least one of the velocity and the rate of the fluid flow in the second direction.

25. The monitor of claim 15, wherein the processor measures the reflux flow of fluid based on the signal output by the sensor.

26. The monitor of claim 15, wherein the monitor comprises a capsule.

27. The monitor of claim 15, further comprising an attachment mechanism to attach the housing to a mucosal lining of the esophagus.

28. The monitor of claim 27, further comprising a chamber to receive tissue of the mucosal lining, wherein the attachment mechanism comprises a pin that advances through the tissue.

29. The monitor of claim 15, further comprising a transmitter, wherein the processor transmits flow information to a receiver located outside a body of a patient.

30. A medical monitor comprising:
a housing;
an attachment mechanism to attach the housing to a mucosal lining of a gastrointestinal tract; and
a flow sensor to sense fluid flow outside of the housing within the gastrointestinal tract.

31. The monitor of claim 30, wherein the sensor comprises an ultrasonic flow sensor.

32. The monitor of claim 30, wherein the sensor comprises an optical flow sensor.

33. The monitor of claim 30, wherein the sensor comprises at least one of an electromagnetic flow sensor, a temperature sensor, and a sensor that senses concentration of a component of the fluid.

34. The monitor of claim 30, further comprising a processor to monitor fluid flow based on a signal output by the sensor as a function of the fluid flow.

35. The monitor of claim 34, wherein the sensor outputs the signal as a function of at least one of velocity and rate of fluid flow.

36. The monitor of claim 34, wherein the processor measures fluid flow based on the signal.

37. The monitor of claim 34, further comprising a transmitter, wherein the processor transmits flow information to a receiver located outside of a patient via the transmitter.

38. The monitor of claim 34, wherein the housing is attached to a mucosal lining of an esophagus, and the processor monitors reflux flow of fluid from a stomach to the esophagus.

39. The monitor of claim 30, further comprising a chamber to receive tissue of the mucosal lining, wherein the attachment mechanism comprises a pin that advances through the tissue.

40. The monitor of claim 30, wherein the attachment mechanism is biodegradable.

41. A medical monitor comprising:
means for housing components of the medical monitor;
means for attaching the housing means to a mucosal lining of a gastrointestinal tract of a patient; and
means for monitoring fluid flow outside of the housing within the gastrointestinal tract.

42. The medical monitor of claim 41, wherein means for monitoring fluid flow comprises means for monitoring reflux flow of fluid from a stomach to an esophagus.

43. The medical monitor of claim 42, further comprising means for determining a direction of the fluid flow, wherein the means for monitoring comprises means for monitoring reflux flow of fluid from a stomach to an esophagus based on the direction.

44. The medical monitor of claim 41, wherein the means for attaching is biodegradable.

45. A system comprising:
a monitor that includes a housing, an attachment mechanism to attach the housing to a mucosal lining of a gastrointestinal tract, and a flow sensor to sense fluid flow within the gastrointestinal tract outside of the housing; and
a receiver to receive fluid flow measurement information from the monitor.

46. The system of claim 45, further comprising a delivery device to carry the monitor to a location within the gastrointestinal tract for attachment at the location.

47. The system of claim 46, wherein the delivery device includes a lumen for application of suction to the lining of the gastrointestinal tract.

48. A method for monitoring fluid flow within the gastrointestinal tract of a patient using a monitor that comprises a housing and a flow sensor, the method comprising:
guiding the monitor to an attachment site within the gastrointestinal tract;
attaching the monitor to a mucosal lining of the gastrointestinal tract at the attachment site; and
monitoring fluid flow within the gastrointestinal tract based on a signal output by the sensor.

49. The method of claim 48, wherein the attachment site is within the esophagus, and monitoring fluid flow comprises monitoring the reflux flow of fluid from the stomach into the esophagus based on the signal.

50. The method of claim 49, wherein monitoring the reflux flow comprises determining a direction of the fluid flow within the esophagus based on the signal, and identifying reflux flow of fluid comprises identifying reflux flow of fluid based on the direction of the fluid flow.

51. The method of claim 49, wherein the attachment site is located proximate to a lower esophageal sphincter of the patient.

52. The method of claim 49, further comprising diagnosing gastroesophageal reflux disease based on identification of reflux flow of fluid from the stomach into the esophagus.

53. The method of claim 48, wherein guiding the monitor to an attachment site comprises guiding the monitor to the attachment site via an endoscopic delivery device.
